(19) United States
(12) Patent Application Publication
(10) Pub. No.: US 2025/0111904 A1
(43) Pub. Date: Apr. 3, 2025

(54) COMPUTER-READABLE RECORDING MEDIUM STORING GROUND ENERGY CALCULATION PROGRAM, GROUND ENERGY CALCULATION DEVICE, AND GROUND ENERGY CALCULATION METHOD

(71) Applicant: Fujitsu Limited, Kawasaki-shi (JP)

(72) Inventor: Naoki IIJIMA, Kawasaki (JP)

(73) Assignee: Fujitsu Limited, Kawasaki-shi (JP)

(21) Appl. No.: 18/820,490

(22) Filed: Aug. 30, 2024

(30) Foreign Application Priority Data
Oct. 3, 2023 (JP) 2023-172295

(51) Int. Cl. G16C 20/20

(52) U.S. Cl. CPC G16C 20/20

(57) ABSTRACT

A non-transitory computer-readable recording medium stores a ground energy calculation program for causing a computer to execute processing including: dividing a target molecule into a plurality of fragments; calculating an energy value of each of a plurality of bath orbitals that corresponds to the fragment, for each divided fragment; selecting a predetermined number of first energy values from a plurality of the energy values, based on the calculated energy value; adding a first bath orbital that corresponds to the first energy value, of the plurality of bath orbitals, to the fragment; calculating energy of the fragment that includes the first bath orbital; and adding the energy calculated for each fragment so as to calculate ground energy of the target molecule.

### CROSS-REFERENCE TO RELATED APPLICATION

This application is based upon and claims the benefit of priority of the prior Japanese Patent Application No. 2023-172295, filed on Oct. 3, 2023, the entire contents of which are incorporated herein by reference.

### FIELD

The embodiment discussed herein is related to a ground energy calculation technology.

### BACKGROUND

By obtaining energy of a target molecule, it is possible to grasp characteristics of the molecule. For example, a state where a molecule structure is stable can be grasped from ground energy of the target molecule, and a state where the molecule structure is unstable can be grasped from excitation energy. Since grasping of such molecule characteristics is useful for drug discovery, discovery of new materials, or the like, quantum chemical calculation is highly important.

Japanese National Publication of International Patent Application No. 2019-537129 is disclosed as related art.

### SUMMARY

According to an aspect of the embodiments, a non-transitory computer-readable recording medium stores a ground energy calculation program for causing a computer to execute processing including: dividing a target molecule into a plurality of fragments; calculating an energy value of each of a plurality of bath orbitals that corresponds to the fragment, for each divided fragment; selecting a predetermined number of first energy values from a plurality of the energy values, based on the calculated energy value; adding a first bath orbital that corresponds to the first energy value, of the plurality of bath orbitals, to the fragment; calculating energy of the fragment that includes the first bath orbital; and adding the energy calculated for each fragment so as to calculate ground energy of the target molecule.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram for explaining a VQE;

FIG. 2 is a diagram illustrating an example of quantum chemical calculation using the VQE;

FIG. 3 is a diagram for explaining a problem of VQE execution in a noisy environment;

FIG. 4 is a diagram for explaining a density matrix embedding theory (DMET);

FIG. 5 is a diagram illustrating an example of a correlation relationship between the number of bath orbitals and expected value calculation accuracy of the VQE;

FIG. 6 is a diagram illustrating a configuration example of a ground energy calculation device 10 according to the present embodiment;

FIG. 7 is a flowchart illustrating a flow of ground energy calculation processing according to the present embodiment;

FIG. 8 is a flowchart illustrating a flow of bath orbital addition processing according to the present embodiment; and

FIG. 9 is a diagram illustrating a hardware configuration example of the ground energy calculation device 10 according to the present embodiment.

### DESCRIPTION OF EMBODIMENTS

Currently, development of a quantum computer that performs the quantum chemical calculation has been progressing with a fault tolerant quantum computer (FTQC) that is a quantum computer with quantum error tolerance, as a target. On the other hand, it is beginning to be said that a noisy intermediate-scale quantum computer (NISQ) with insufficient quantum error correction can also realize superiority of the quantum computer in fields of quantum chemical calculation, quantum approximation optimization, quantum machine learning, or the like.

The NISQ is an intermediate-scale quantum computer having noise. The NISQ has a limit on a time in which a quantum state can be maintained. Therefore, for example, the NISQ can execute only a shallow quantum circuit of which the number of quantum bits is several tens to several hundreds, or the like, for example, a small-scale quantum circuit.

Furthermore, a current NISQ is easily affected by the noise, an execution result thereof tends to easily vary due to the noise. A magnitude of the influence of the noise depends on a size of the quantum circuit (the numbers of quantum bits and gates), and the number of gates tends to increase as the number of quantum bits increases.

Furthermore, as one variational algorithm for the NISQ, there is a variational quantum eigensolver (VQE). The VQE is an algorithm that can search for a ground state using a quantum state in the quantum chemical calculation and calculate ground energy.

However, accuracy of expected value calculation of the VQE deteriorates due to an influence of noise. Moreover, a larger molecule to be handled requires a larger quantum circuit. Therefore, the influence of the noise may be increased, and a degree of the deterioration in the accuracy of the VQE increases. The accuracy deterioration of the VQE also affects accuracy when the NISQ calculates ground energy of a molecule using the VQE.

In one aspect, an object is to accurately calculate ground energy of a molecule.

Hereinafter, examples of a ground energy calculation program, a ground energy calculation device, and a ground energy calculation method according to the present embodiment will be described in detail with reference to the drawings. Note that the present embodiment is not limited by these examples. Furthermore, the respective examples may be appropriately combined within a range without inconsistency.

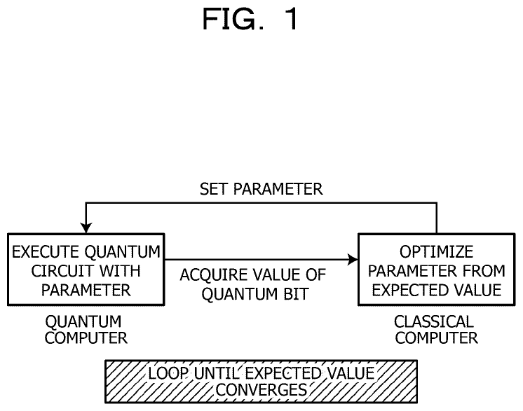

First, a VQE used in the present embodiment will be described. FIG. 1 is a diagram for explaining the VQE. The VQE is, for example, a variational algorithm for a NISQ, and as illustrated in FIG. 1, is executed by a method alternately using the NISQ (quantum computer) and a classical computer that is a general computer currently used.

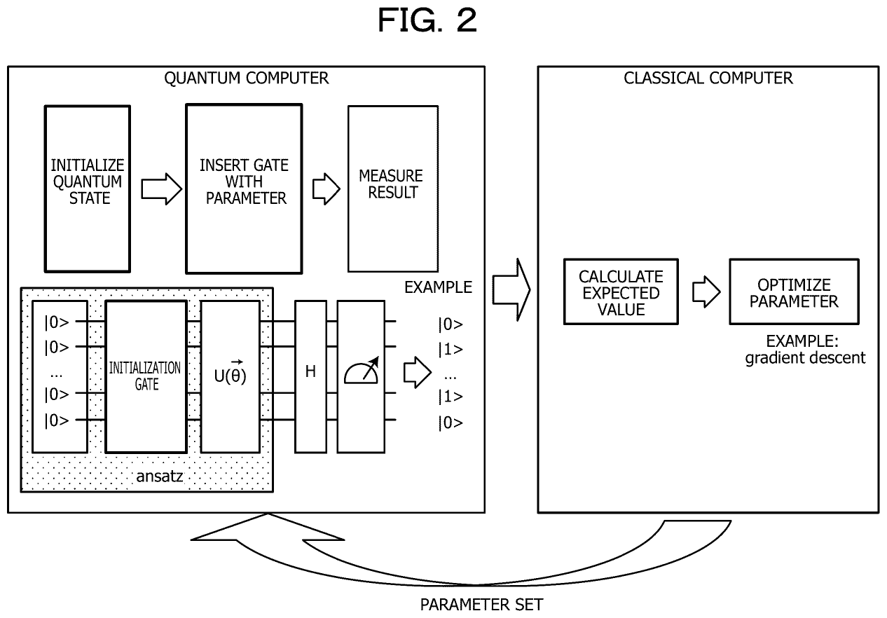

In the method using the VQE, for example, as illustrated in FIG. 1, the quantum computer executes a quantum circuit with a parameter, and the classical computer acquires a value of a quantum bit that is an execution result. Next, for example, the classical computer calculates an expected value by execution of the quantum circuit, based on the acquired value of the quantum bit, and optimizes the parameter from the expected value. Then, for example, as illustrated in FIG. 1, until the optimized parameter is set to the quantum circuit and the expected value converges, the execution of the quantum circuit and the optimization of the parameter are repeated. This will be specifically described with reference to FIG. 2.

FIG. 2 is a diagram illustrating an example of quantum chemical calculation using the VQE. FIG. 2 is an example in which the quantum computer executes ansatz that is a quantum circuit with a parameter and the classical computer optimizes the parameter.

The quantum circuit illustrated in FIG. 2 includes, for example, an initialization gate that initializes a quantum state of each quantum bit, a gate group U (θ) (parameter θ is correctly described by writing→above θ) used to construct a trial state, and H obtained by gate-converting a Hamiltonian to be calculated. As illustrated in FIG. 2, for example, the quantum computer initializes the quantum state of each quantum bit, inserts the quantum state into a gate with a parameter, and measures a value of each quantum bit, for example, the quantum state, as an execution result.

Next, for example, as illustrated in FIG. 2, the classical computer calculates an expected value from the measured value of each quantum bit and optimizes the parameter, using gradient descent or the like, that is an existing optimization method, based on the expected value. Then, for example, until the optimized parameter is set to the quantum circuit and the expected value converges, the execution of the quantum circuit and the optimization of the parameter are repeated.

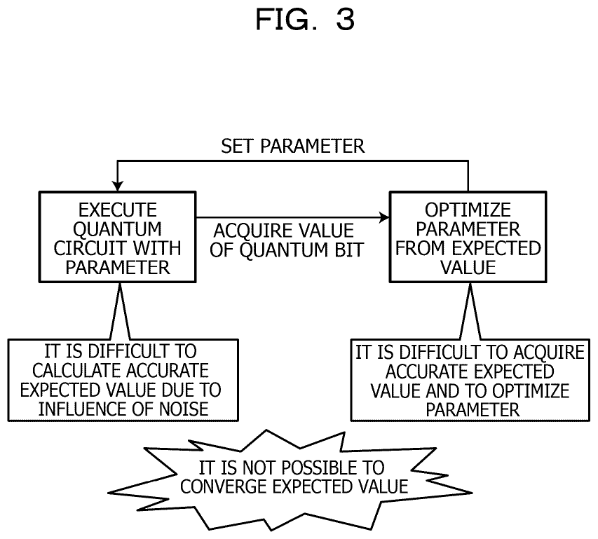

However, for example, the execution of the quantum circuit using the VQE has a problem that accuracy of expected value calculation deteriorates due to an influence of noise. FIG. 3 is a diagram for explaining a problem of VQE execution in a noisy environment. As illustrated in FIG. 3, for example, it is difficult to accurately calculate the expected value, and in addition, to optimize the parameter, due to the influence of the noise. Therefore, the expected value does not converge, and the accuracy of the quantum circuit execution using the VQE deteriorates. Furthermore, when a larger molecule to be handled requires a larger quantum circuit. Therefore, the influence of the noise may be increased, and a degree of the deterioration in the accuracy of the quantum circuit execution using the VQE increases. Such deterioration in the accuracy of the quantum circuit execution using the VQE also affects accuracy when the NISQ calculates ground energy of the molecule using the VQE.

Therefore, one of the objects of the present embodiment is, for example, to reduce the number of quantum bits of the quantum circuit used in the VQE, to reduce the influence of the noise on the VQE, and to accurately calculate the ground energy of the molecule.

Therefore, in the present embodiment, the molecule to be handled is divided into a plurality of fragments, using the density matrix embedding theory (DMET), which is an existing technology. The DMET is an existing method for dividing a molecule into a plurality of fragments, summing energy of each fragment, and obtaining energy of the entire molecule. By dividing the molecule into the plurality of fragments, it is possible to reduce the number of orbitals of each fragment as compared with the entire molecule, and it is possible to reduce the number of quantum bits.

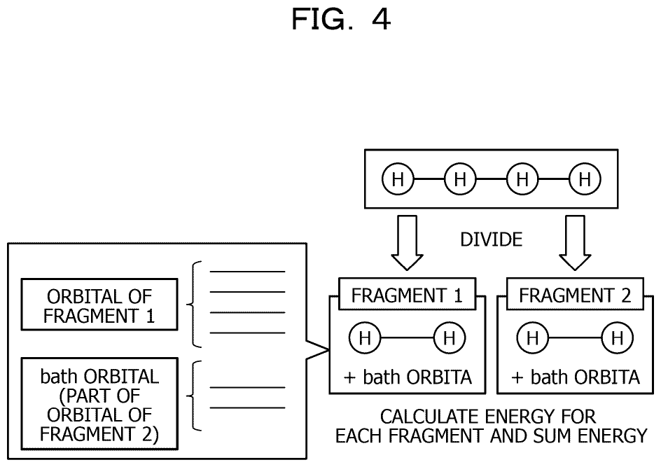

FIG. 4 is a diagram for explaining the DMET. As illustrated in FIG. 4, in the DMET, for example, the molecule is divided into the plurality of fragments. In the example in FIG. 4, four molecules are divided into two fragments 1 and 2, each including two molecules. For example, each fragment includes an orbital of each fragment and a bath orbital that is a part of an orbital of another fragment. The orbital of the fragment is, for example, an orbital in which electrons can enter. Furthermore, the bath orbital represents, for example, an interaction between the fragments. For example, as illustrated in FIG. 4, the fragment 1 includes an orbital of the fragment 1 and the bath orbital that is a part of an orbital of the fragment 2. Then, energy of the entire molecule can be calculated by calculating energy for each fragment and summing the calculated energy.

Furthermore, for example, in the DMET, the number of quantum bits required for the calculation of the fragment is determined based on the number of orbitals of the fragment+the number of bath orbitals. Furthermore, in the DMET, for example, by dividing the molecule, it is possible to reduce the number of orbitals of each fragment, as compared with the entire molecule. Therefore, it is possible to reduce the number of quantum bits of the quantum circuit used in the VQE and to reduce the influence of the noise on the VQE. However, since the number of quantum bits corresponding to the number of bath orbitals is required, the number of bath orbitals included in the fragment is further adjusted in the present embodiment. Although details of the adjustment of the number of bath orbitals will be described later, for example, only top N (N is an arbitrary natural number) bath orbitals having a large influence on the interaction between the fragments is included in the fragment. As a result, it is possible to reduce the number of quantum bits of the fragment and to reduce the influence of the noise on the VQE, for example, the expected value calculation of the VQE.

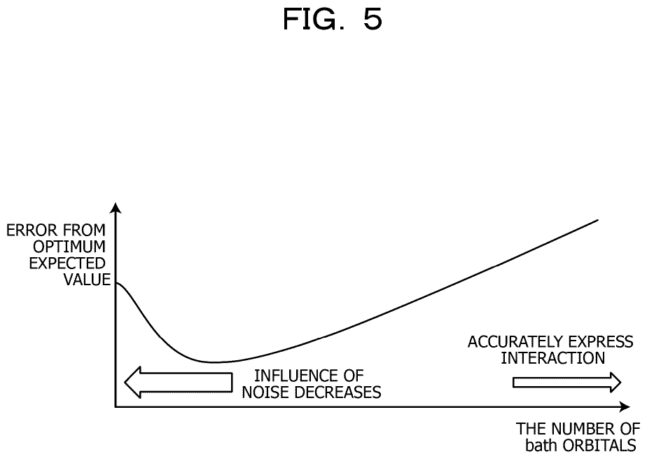

FIG. 5 is a diagram illustrating an example of a correlation relationship between the number of bath orbitals and expected value calculation accuracy of the VQE. As illustrated in FIG. 5, for example, as the number of bath orbitals is larger, the interaction between the fragments can be more accurately expressed. However, when the number of bath orbitals increases, the number of quantum bits increases. Therefore, the influence of the noise increases, and an error from an optimum expected value increases, and the accuracy deteriorates. Conversely, for example, when the number of bath orbitals decreases, the influence of the noise decreases. However, when the influence of the noise becomes too small, it is not possible to express the interaction between the fragments, the error from the optimum expected value increases, and the accuracy deteriorates. Therefore, as illustrated in FIG. 5, by adjusting the number of bath orbitals to such an extent that the interaction between the fragments can be expressed and to be a smaller number, the error from the optimum expected value is reduced, and the accuracy can be improved.

Therefore, in the present embodiment, by dividing the molecule to be handled into the plurality of fragments and adjusting the number of bath orbitals included in each fragment, using the DMET, the influence of the noise on the expected value calculation of the VQE is reduced, and thus, the ground energy of the molecule is accurately calculated.

### Functional Configuration of Ground Energy Calculation Device 10

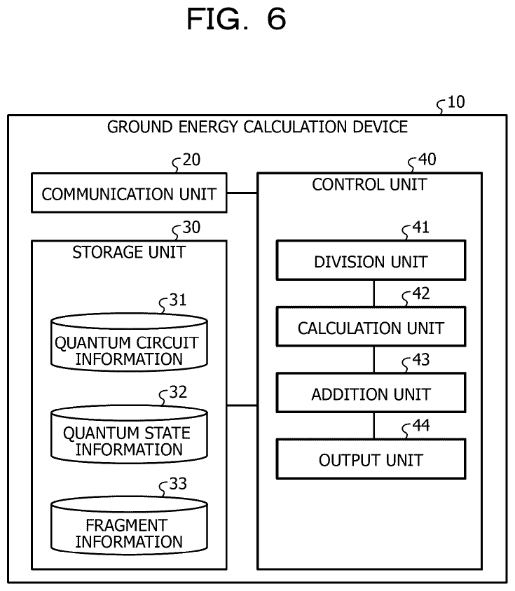

Next, a functional configuration of a ground energy calculation device 10 to be an operation subject of the present embodiment will be described. FIG. 6 is a diagram illustrating a configuration example of the ground energy calculation device 10 according to the present embodiment. The ground energy calculation device 10 illustrated in FIG. 6 is, for example, a quantum computer such as a NISQ and is an information processing device such as a server computer, a desktop personal computer (PC), or a notebook PC. Note that FIG. 6 illustrates the ground energy calculation device 10 as one computer. However, the ground energy calculation device 10 may be a distributed computing system including a plurality of computers. Furthermore, for example, a part or all of the ground energy calculation device 10 may be a cloud computer device managed by a service provider that provides a cloud computing service.

As illustrated in FIG. 6, the ground energy calculation device 10 includes, for example, a communication unit 20, a storage unit 30, and a control unit 40.

The communication unit 20 is a processing unit that controls communication with another device, and is a communication interface such as a network interface card, or a universal serial bus (USB) interface, for example.

The storage unit 30 has a function of storing various types of data and a program that the control unit 40 executes, and is implemented by a storage device such as a memory or a hard disk, for example. The storage unit 30 stores quantum circuit information 31, quantum state information 32, fragment information 33, or the like.

In the quantum circuit information 31, for example, information regarding a quantum circuit executed by the ground energy calculation device 10 or the like is stored. For example, in the quantum circuit information 31, for example, information regarding the quantum circuit with the parameter as illustrated in FIG. 2 is stored.

In the quantum state information 32, for example, information regarding a quantum state that is a state of a quantum bit input when the ground energy calculation device 10 executes the quantum circuit or the like is stored. Furthermore, in the quantum state information 32, for example, information regarding a quantum state that is a state of a quantum bit output from the quantum circuit executed by the ground energy calculation device 10 or the like may be stored.

In the fragment information 33, for example, information regarding the fragment divided using the DMET or the like is stored. For example, in the fragment information 33, the orbital of each fragment, the bath orbital, the number of orbitals of each fragment and the number of bath orbitals, energy and the number of electrons calculated for each fragment, or the like may be stored.

Note that the information to be stored in the storage unit 30 described above is merely an example, and the storage unit 30 may store various types of information other than the information described above.

The control unit 40 is a processing unit that controls the entire ground energy calculation device 10 and is, for example, a processor or the like. The control unit 40 includes a division unit 41, a calculation unit 42, an addition unit 43, and an output unit 44. Note that each processing unit is an example of an electronic circuit that a processor includes, or an example of a process that the processor performs.

The division unit 41 divides a target molecule into a plurality of fragments, for example, using the DMET.

The calculation unit 42 calculates, for example, an energy value of each of the plurality of bath orbitals corresponding to the fragment, for each fragment divided by the division unit 41. Regarding the calculation of the bath orbital, for example, the calculation unit 42 calculates a one-electron reduced density matrix (1-RDM) of the entire target molecule. Note that the density matrix is, for example, a matrix representing a quantum state. Then, for example, the calculation unit 42 generates a matrix, excluding the orbital included in the fragment, from the calculated 1-RDM, diagonalizes the generated matrix, and calculates an eigenvalue of the matrix as the energy value of the bath orbital.

Furthermore, for example, the calculation unit 42 calculates energy of a fragment including a first bath orbital added by the addition unit 43 and adds the energy calculated for each fragment, so as to calculate the ground energy of the target molecule.

For example, the addition unit 43 selects a predetermined number of first energy values from among the plurality of energy values, based on the energy value of each of the plurality of bath orbitals, calculated by the calculation unit 42. Note that, in the selection of the predetermined number of first energy values, for example, the predetermined number of first energy values may be selected in the descending order of a value of the energy value of each of the plurality of bath orbitals.

Furthermore, for example, the addition unit 43 adds the first bath orbital corresponding to the first energy value, among the plurality of bath orbitals, to the fragment. For example, the bath orbitals other than the first bath orbital, of the plurality of bath orbitals are not added to the fragment, and quantum bits therefor can be reduced.

The output unit 44 outputs, for example, the ground energy of the target molecule calculated by the calculation unit 42. For example, the output of the ground energy may be display via a display of the ground energy calculation device 10 or the like or may be transmission to the classical computer communicably coupled to the ground energy calculation device 10 or the like.

### Flow of Processing

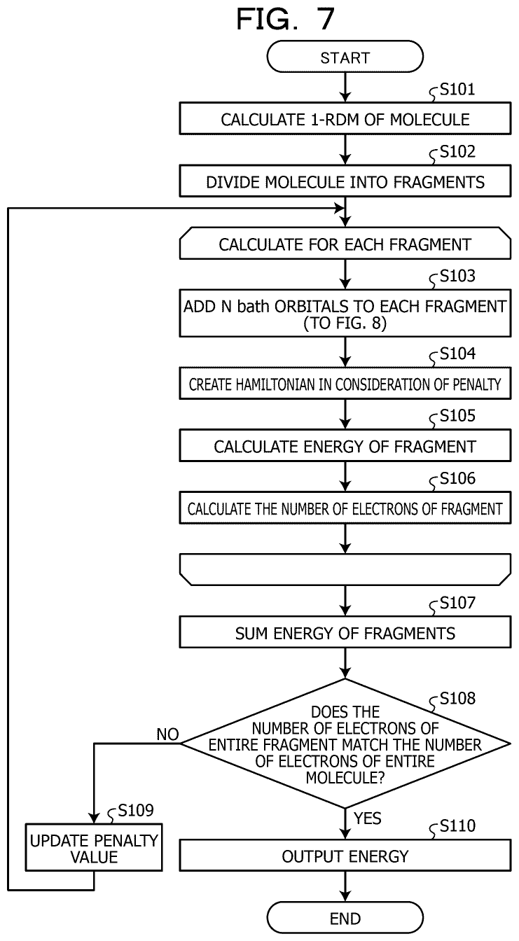

Next, ground energy calculation processing executed by the ground energy calculation device 10 will be described, with reference to FIG. 7. FIG. 7 is a flowchart illustrating a flow of the ground energy calculation processing according to the present embodiment.

First, as illustrated in FIG. 7, the ground energy calculation device 10 calculates, for example, the 1-RDM of the entire target molecule (step S101).

Next, for example, the ground energy calculation device 10 divides the target molecule into the plurality of fragments, using the DMET (step S102). Note that subsequent processing in steps S103 to S106 is executed for each fragment divided in step S102.

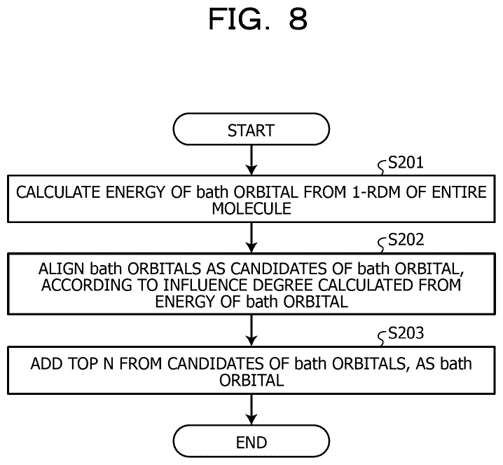

Next, for example, the ground energy calculation device 10 adds N bath orbitals to the fragment (step S103). An example of bath orbital addition processing in step S103 will be more specifically described, with reference to FIG. 8.

FIG. 8 is a flowchart illustrating a flow of the bath orbital addition processing according to the present embodiment. First, as illustrated in FIG. 8, for example, the ground energy calculation device 10 calculates the energy of the bath orbital from the 1-RDM of the entire target molecule calculated in step S101 (step S201). For example, the ground energy calculation device 10 generates the matrix, excluding the orbital included in the fragment, from the calculated 1-RDM, diagonalizes the generated matrix, and calculates the eigenvalue of the matrix as the energy value of the bath orbital.

Next, the ground energy calculation device 10 aligns the bath orbitals as candidates of the bath orbital, according to an influence degree on the interaction between the fragments calculated from the energy of the bath orbital calculated in step S201 (step S202). Note that the candidate of the bath orbital is, for example, a candidate of the bath orbital to be added to the fragment.

Next, for example, the ground energy calculation device 10 adds top N bath orbitals, from among the candidates of the bath orbitals aligned in step S202, to the fragment, as official bath orbitals to be adopted (step S203). For example, by including only the top N bath orbitals that have a large influence on the interaction between the fragments in the fragment, it is possible to reduce the number of quantum bits of the fragment, and it is possible to reduce the influence of the noise on the expected value calculation of the VQE. After the execution of step S203, the bath orbital addition processing illustrated in FIG. 8 ends, and the procedure returns to step S103 of the ground energy calculation processing illustrated in FIG. 7.

Next, for example, the ground energy calculation device 10 creates a Hamiltonian, in consideration of a penalty (step S104).

Next, for example, the ground energy calculation device 10 calculates the energy of the fragment to which the bath orbital is added in step S103 (step S105).

Next, for example, the ground energy calculation device 10 calculates the number of electrons of the fragment to which the bath orbital is added in step S103 (step S106). The ground energy calculation device 10 executes the processing in steps S103 to S106 for each fragment divided in step S102. After the processing is executed on all the fragments divided in step S102, the procedure proceeds to step S107.

Next, for example, the ground energy calculation device 10 sums the energy of the respective fragments calculated in step S105 (step S107).

Next, for example, the ground energy calculation device 10 sums the number of electrons of each fragment calculated in step S106, sets the calculated number as the number of electrons of the entire fragment, and determines whether or not the number of electrons matches the number of electrons of the entire target molecule (step S108). In a case where both numbers of electrons do not match (step S108: No), the ground energy calculation device 10 updates, for example, a penalty value (step S109), and executes the processing again from step S103.

On the other hand, in a case where both numbers of electrons match (step S108: Yes), for example, the ground energy calculation device 10 outputs the energy summed in step S107 as the ground energy of the target molecule (step S110). After the execution of step S110, the ground energy calculation processing illustrated in FIG. 7 ends.

### Effects

As described above, the ground energy calculation device 10

divides the target molecule into the plurality of fragments, calculates the energy value of each of the plurality of bath orbitals corresponding to the fragments, for each divided fragment, selects the predetermined number of first energy values from the plurality of energy values, based on the calculated energy values, adds the first bath orbital corresponding to the first energy value, of the plurality of bath orbitals, to the fragment, calculates the energy of the fragment including the first bath orbital, and adds the energy calculated for each fragment, so as to calculate the ground energy of the target molecule.

In this way, the ground energy calculation device 10 adds the predetermined number of bath orbitals to the fragment, based on the energy of the bath orbital calculated for each fragment obtained by dividing the target molecule and sums the energy of each fragment. As a result, the ground energy calculation device 10 can accurately calculate the ground energy of the molecule.

Furthermore, the processing for selecting the first energy value, executed by the ground energy calculation device 10 includes processing for selecting the predetermined number of first energy values in the descending order of a value of the calculated energy value.

As a result, the ground energy calculation device 10 can accurately calculate the ground energy of the molecule.

Furthermore, the ground energy calculation device 10 calculates the 1-RDM of the entire target molecule, and the processing for calculating the energy value of each of the plurality of bath orbitals, executed by the ground energy calculation device 10 includes processing for generating the matrix excluding the orbital included in the fragment, from the calculated 1-RDM, diagonalizing the generated matrix, and calculating the eigenvalue of the matrix as the energy value.

As a result, the ground energy calculation device 10 can accurately calculate the ground energy of the molecule.

### System

Pieces of information including the processing procedures, the control procedures, the specific names, the various types of data, and the parameters described above or illustrated in the drawings may be changed as appropriate, unless otherwise specified. Furthermore, the specific examples, the distributions, the numerical values, and the like described in the example are merely examples, and may be changed as appropriate.

Furthermore, specific forms of separation and integration of the components of the ground energy calculation device 10 are not limited to those illustrated in the drawings. For example, the calculation unit 42 of the ground energy calculation device 10 may be distributed to a plurality of processing units, or the calculation unit 42 and the addition unit 43 of the ground energy calculation device 10 may be integrated into one processing unit. For example, all or some of the components may be functionally or physically separated or integrated in optional units, depending on various types of loads, use situations, or the like. Moreover, all or any part of the respective processing functions of the respective devices may be implemented by a central processing unit (CPU) and a program analyzed and executed by the CPU, or may be implemented as hardware by wired logic.

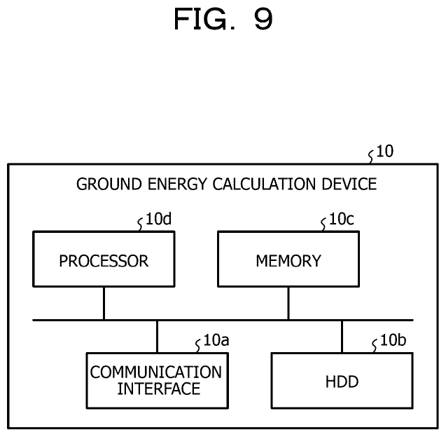

FIG. 9 is a diagram illustrating a hardware configuration example of the ground energy calculation device 10 according to the present embodiment. As illustrated in FIG. 9, the ground energy calculation device 10 includes a communication interface 10a, a hard disk drive (HDD) 10b, a memory 10c, and a processor 10d. Furthermore, the individual components illustrated in FIG. 9 are coupled to each other by a bus or the like.

The communication interface 10a is a network interface card or the like, and communicates with another information processing device. The HDD 10b stores a program and data for operating each function of the ground energy calculation device 10, for example.

The processor 10d is a CPU, a micro processing unit (MPU), a graphics processing unit (GPU), or the like. In addition, the processor 10d may be implemented by an integrated circuit such as an application specific integrated circuit (ASIC), or a field programmable gate array (FPGA). The processor 10d reads, from the HDD 10b or the like, a program for executing processing similar to that of each processing unit illustrated in FIG. 6 or the like, and loads the program into the memory 10c, for example. As a result, the processor 10d can operate as a hardware circuit that performs a process for implementing each function of the ground energy calculation device 10.

Furthermore, the ground energy calculation device 10 can implement functions similar to the functions of the above example by reading the above program from a recording medium with a medium reading device and executing the above read program. Note that other programs referred to in the example are not limited to being executed by the ground energy calculation device 10. For example, the above example may also be applied in a case where another information processing device executes the program, or a case where another information processing device and the ground energy calculation device 10 cooperate to execute the program.

The program may be distributed via a network such as the Internet. Alternatively, the program may be recorded in a computer-readable recording medium such as a hard disk, a flexible disk (FD), a compact disc read only memory (CD-ROM), a magneto-optical disk (MO), or a digital versatile disc (DVD). Then, the program may be read from the recording medium and be executed by the ground energy calculation device 10 or the like.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a ground energy calculation program for causing a computer to execute processing comprising:
dividing a target molecule into a plurality of fragments;
calculating an energy value of each of a plurality of bath orbitals that corresponds to the fragment, for each divided fragment;
selecting a predetermined number of first energy values from a plurality of the energy values, based on the calculated energy value;
adding a first bath orbital that corresponds to the first energy value, of the plurality of bath orbitals, to the fragment;
calculating energy of the fragment that includes the first bath orbital; and
adding the energy calculated for each fragment so as to calculate ground energy of the target molecule.

2. The non-transitory computer-readable recording medium according to claim 1, wherein
the processing of selecting the first energy value includes
processing of selecting the predetermined number of first energy values in descending order of a value of the calculated energy value.

3. The non-transitory computer-readable recording medium according to claim 1, for causing the computer to execute processing comprising:
calculating a one-electron reduced density matrix (1-RDM) of the entire target molecule, wherein
the processing of calculating the energy value of each of the plurality of bath orbitals includes processing of
generating a matrix that excludes an orbital included in the fragment, from the calculated 1-RDM,
diagonalizing the generated matrix, and calculating an eigenvalue of the matrix as the energy value.

4. A ground energy calculation device comprising:
a memory; and
a processor coupled to the memory and configured to:
divide a target molecule into a plurality of fragments;
calculate an energy value of each of a plurality of bath orbitals that corresponds to the fragment, for each divided fragment;
select a predetermined number of first energy values from a plurality of the energy values, based on the calculated energy value;
add a first bath orbital that corresponds to the first energy value, of the plurality of bath orbitals, to the fragment;
calculate energy of the fragment that includes the first bath orbital; and
add the energy calculated for each fragment so as to calculate ground energy of the target molecule.

5. The ground energy calculation device according to claim 4, wherein the processor selects the predetermined number of first energy values in descending order of a value of the calculated energy value.

6. The ground energy calculation device according to claim 4, wherein the processor:
calculates a one-electron reduced density matrix (1-RDM) of the entire target molecule;
generates a matrix that excludes an orbital included in the fragment, from the calculated 1-RDM;
diagonalizes the generated matrix; and
calculates an eigenvalue of the matrix as the energy value.

7. A ground energy calculation method for causing a computer to execute processing comprising:
dividing a target molecule into a plurality of fragments;
calculating an energy value of each of a plurality of bath orbitals that corresponds to the fragment, for each divided fragment;
selecting a predetermined number of first energy values from a plurality of the energy values, based on the calculated energy value;
adding a first bath orbital that corresponds to the first energy value, of the plurality of bath orbitals, to the fragment;
calculating energy of the fragment that includes the first bath orbital; and
adding the energy calculated for each fragment so as to calculate ground energy of the target molecule.

8. The ground energy calculation method according to claim 7, wherein
the processing of selecting the first energy value includes
processing of selecting the predetermined number of first energy values in descending order of a value of the calculated energy value.

9. The ground energy calculation method according to claim 7, comprising:
calculating a one-electron reduced density matrix (1-RDM) of the entire target molecule, wherein
the processing of calculating the energy value of each of the plurality of bath orbitals includes processing of
generating a matrix that excludes an orbital included in the fragment, from the calculated 1-RDM,
diagonalizing the generated matrix, and calculating an eigenvalue of the matrix as the energy value.
